(19) United States
(12) Patent Application Publication
(10) Pub. No.: US 2020/0205794 A1
(43) Pub. Date: Jul. 2, 2020

(54) A TISSUE SAMPLE PUNCH

(71) Applicant: SNPSHOT TRUSTEE LIMITED, Auckland (NZ)

(72) Inventors: Roy Victor BLADEN, Auckland (NZ); Michael Stuart GARDNER, Auckland (NZ)

(21) Appl. No.: 16/612,712

(22) PCT Filed: May 11, 2018
(86) PCT No.: PCT/IB2018/053281

§ 371 Date: Nov. 11, 2019

(30) Foreign Application Priority Data
May 11, 2017 (NZ) 731818

(51) Int. Cl. A61B 10/02; A61B 17/3205

(52) U.S. Cl. CPC A61B 10/0275; A01K 11/003; A61B 17/32053

(57) ABSTRACT

A tissue sample punch to puncture into and through tissue, the punch comprising a tubular body having a tissue puncturing end and a trailing end, the tissue puncturing end being formed to define saddle cut having at least two peaks that define the distal extent of the puncturing end.

### CROSS-REFERENCE TO RELATED APPLICATION(S)

This application is a National Stage Entry of Application No.: PCT/IB2018/053281 filed May 11, 2018, which claims benefit of priority of New Zealand Patent Application No. 731818, filed on May 11, 2017. The entire contents of the earlier application are incorporated herein by reference.

### FIELD OF THE INVENTION

The present invention relates to a punch for taking biological samples, such as but not limited to samples of animal tissue.

### BACKGROUND TO THE INVENTION

When raising and caring for animals it may be desirable to create and maintain biological records of the animals. One method of obtaining such biological records is by the taking of samples of tissue of the animal, which may be analyzed to obtain information about the animal, such as its genetic makeup or its health. These records may periodically be taken over the lifetime of an animal, particularly in the case of production animals such as cattle.

One common form of such a tissue sample is of a biopsy sample of tissue from the ear of the animal. In fish, a common form of such a tissue sample may be a biopsy sample of tissue from a fin of the fish.

It may also be desirable to take other forms of biological samples for maintaining biological records, such as taking samples of plants. This could be a biopsy or non-biopsy sample, of material such as leaves, bark, roots, etc.

It is an object of the present invention to provide an improved punch for taking a sample of animal tissue, or at least to provide the public with a useful choice.

Where used in this specification tissue means any part of a living thing, particularly any part made up of similar cells, or any part or parts that perform a similar function. Tissue preferably refers to any form of biological sample, from plants and animals particularly, including pigs, goats, cattle, sheep, poultry, and fish. Biological samples may include for example, animal tissue such as flesh, blood, hair, fur, saliva, sweat, urine, etc, or plant tissue such as leaves, bark, roots or wood, or any other part of a plant or animal but particularly those that are made up of similar cells, or which perform a similar function.

The present invention may be used at least for either or both of production animals and companion animals. It is anticipated that production animals may include but not be limited to bovine, pigs, deer and sheep. Further it is anticipated that companion animals may include but not be limited to horses, cats and dogs.

### SUMMARY OF THE INVENTION

In a first aspect, the present invention broadly consists in a tissue sample punch to puncture into and through animal tissue (e.g. skin, flesh and hair) of an animal, the punch comprising

a tubular body having a tissue puncturing end and a trailing end, the tissue puncturing end being formed to define saddle cut having at least two peaks that define the distal end of the puncturing end.

In a further aspect, the present invention broadly consists in a tissue sample punch comprising a tubular punch body having a cutting end and a free end, the cutting end comprising a cutting edge formed and having a perimeter greater than the circumference of the tubular punch body at the cutting end, the cutting edge presenting at least one leading portion and one trailing portion.

Preferably the cutting edge at a leading portion comprises a peak in the profile of the cutting edge flanked by wake edges that are at an angle between 40 degrees and 120 degrees to each other.

Preferably the bend of the cutting edge at the leading portion is a sharp bend rather than a curved bend.

Preferably the cutting edge comprises one leading portion, the cutting edge varies continuously in an axial direction of the tubular body from one side of the leading portion to the trailing portion and back to the other side of the leading portion.

Preferably the cutting edge comprises multiple leading portions, the profile of the cutting edge preferably varies continuously in an axial direction of the tubular body between any two leading portions.

Preferably the continuous variation of the cutting edge acts to prevent the tissue being sampled from being caught or snagged on the cutting edge, particularly where the tissue comprises a skin membrane or surface hairs.

Preferably the forming of the cutting edge of the punch is by one of laser cutting, water jet cutting, plasma cutting and mechanical cutting.

Preferably the forming of the cutting edge of the punch is only by one of laser cutting, water jet cutting, plasma cutting and mechanical cutting.

Preferably the tubular punch body at the cutting end comprises a metallic which presents at the cutting edge, subsequent to its forming, a surface finish of greater roughness than the surface finish of the internal and external faces of the tubular punch body.

Preferably the forming causes the cutting edge to comprise a plurality of burrs or serrations.

Preferably the forming of the cutting edge of the punch is by laser cutting.

Preferably the surface finish following the forming by laser cutting comprises a non-continuous surface profile.

Preferably where the forming of the cutting edge is by laser cutting the pulsing of the laser causes the non-continuous surface profile.

Preferably the surface non-continuous surface profile comprises a plurality of burrs or serrations.

Preferably the surface finish acts, during the cutting of a sample, to tear or rip adjacent portions of the tissue to be sampled.

Preferably the cutting edge is not sharpened or de-burred from its post-forming surface finish.

Preferably the burrs or serrations act, during the cutting of a sample, to tear or rip adjacent portions of the tissue to be sampled.

Preferably post-forming the cutting edge remains unsharpened.

Preferably post-forming the burrs or serrations are retained on the cutting edge.

Preferably during the cutting of a tissue sample an item of tissue to be sampled is located with its surface to be penetrated by the punch substantially perpendicular to the length of the tubular punch body.

Preferably during the cutting of a tissue sample the cutting edge makes a first incision at the at least one leading portion from which the cutting edge is progressively engaged with the sample to be cut towards the at least one trailing portion.

Preferably the progressive engagement of the cutting edge, being an edge of length greater than the circumference of the tissue sample to be cut, results in relatively increased shearing or tearing of the tissue at the interface of the cutting edge and the tissue to be sampled.

Preferably between each at least one leading portion and at least one trailing portion the cutting edge has a normal that penetrates the tissue that extends at an angle to the normal of the surface of the tissue it is cutting through.

Preferably the non-perpendicular cutting edge between each at least one leading portion and at least one trailing portion causes a concentration of shear forces on the tissue during the cutting of a tissue sample by the punch.

Preferably during the cutting of a tissue sample having a first and second tissue surfaces, the at least one leading portion of the cutting edge makes the first incision of the cutting edge through each of the first and second tissue surfaces.

Preferably during the cutting of a tissue sample the punch is operated relative to the tissue to be sampled in a purely linear manner.

Preferably where the cutting edge comprises two leading portions the cutting edge is formed by at least one cut such that the wall thickness of the tubular body narrows towards each leading portion.

Preferably the cutting edge is formed by at least one laser cut through at least one wall of the punch body made linearly relative to the tubular body, such that wall thickness of the tubular body varies about the cutting edge.

Preferably during the forming the laser cutting beam moves in a purely linear manner relative to the tubular body.

Preferably the cutting edge is formed by at least one laser cut directed through a wall of the tubular body and perpendicular to the surface of the tubular body, such that tube wall thickness is continuous about cutting edge.

Preferably the cutting edge is formed by at least one laser cut through a wall of the tubular body and directed at a constant angle to the surface of the tubular body, such that the formed cutting edge is at a constant angle relative to the wall of tubular body.

Preferably during the forming the laser cutting beam remains perpendicular or near perpendicular to the surface of the tubular body.

Preferably the forming of the cutting edge comprises cutting at least one V or U shape of material away from the tubular punch body at the cutting end.

Preferably the forming of the cutting edge comprises cutting the outside of a V of material away from the tubular punch body in order to form a saddle profile of the cutting edge, the cutting edge comprising two leading portions and two trailing portions.

Preferably the forming of the cutting edge comprises making at least one U-shaped cut through a wall or walls of the tubular body to form a saddle profile of the cutting edge, the cutting edge comprising two leading portions and two trailing portions.

Preferably the cutting edge comprises two leading portions and two trailing portions, said cutting edge being formed by two laser cuts, each laser cut forming one notch in the tubular body at the cutting end.

Preferably the cutting edge is formed by a single arcuate laser cut, wherein the laser cutting beam is rotated about 180 degrees of the circumference of tubular punch body, the beam passing through a first adjacent wall portion of the tubular punch body and a second opposite wall portion, and wherein the laser cutting beam acts perpendicular or near perpendicular to the surface of the tubular punch body.

Preferably the profile of the cutting edge is of a saddle shape.

Preferably the forming of the cutting edge comprises cutting a saddle cut of material away from the tubular punch body at the cutting end.

Preferably the forming of the cutting edge comprises cutting a fishmouth cut of material away from the tubular punch body at the cutting end.

Preferably the tubular body is of a diameter under 10 mm.

Preferably the tubular body is of a diameter between 2 mm and 5 mm.

Preferably the tubular body is of a diameter between 2.5 mm and 3.5 mm.

Preferably the wall thickness of the tubular body is less than 15% of the diameter of the tubular body.

Preferably the wall thickness of the tubular body is less than 10% of the diameter of the tubular body.

Preferably the wall thickness of the tubular body is between 5% and 10% of the diameter of the tubular body.

Preferably the wall thickness of the tubular body is between 0.1 mm and 1 mm.

A method of manufacturing a tissue sample punch, the method comprising the steps of

providing a tubular body,

forming a cutting edge by making at least one cut through the wall of the tubular body,

wherein the forming is in a manner so as to cause burring or serration of the at least one cut and otherwise unsharpened cutting edge to remain intact.

Preferably the forming of the cutting edge is by at least one cut of one of a laser cutter, water jet cutter, plasma cutter, or mechanical cutter.

Preferably the forming of the cutting edge by at least one cut is such as to provide a cutting edge comprising a plurality of burrs or serrations.

Preferably the burrs or serrations are of a size less than one tenth of the diameter of the tubular punch body.

Preferably the burrs or serrations are of a size less than one twentieth of the diameter of the tubular punch body.

Preferably the step of forming the cutting edge comprises no sharpening or de-burring of the cutting edge, other than any such sharpening or de-burring incidental to the at least one cut.

Preferably the formed cutting edge presents at least one leading portion and at least one trailing portion.

Preferably where the cutting edge comprises two leading portions the cutting edge is formed by at least one cut such that the wall thickness of the tubular body narrows towards each leading portion.

Preferably the cutting edge is formed by at least one laser cut through at least one wall of the punch body made linearly relative to the tubular body, such that wall thickness of the tubular body varies about the cutting edge.

Preferably during the forming the laser cutting beam moves in a purely linear manner relative to the tubular body.

Preferably the cutting edge is formed by at least one laser cut directed through a wall of the tubular body and perpendicular to the surface of the tubular body, such that tube wall thickness is continuous about cutting edge.

Preferably the cutting edge is formed by at least one laser cut through a wall of the tubular body and directed at a constant angle to the surface of the tubular body, such that the formed cutting edge is at a constant angle relative to the wall of tubular body.

Preferably during the forming the laser cutting beam remains perpendicular or near perpendicular to the surface of the tubular body.

Preferably the forming of the cutting edge comprises cutting at least one V or U shape of material away from the tubular punch body at the cutting end.

Preferably the forming of the cutting edge comprises cutting the outside of a V of material away from the tubular punch body in order to form a saddle profile of the cutting edge, the cutting edge comprising two leading portions and two trailing portions.

Preferably the forming of the cutting edge comprises making at least one U-shaped cut through a wall or walls of the tubular body to form a saddle profile of the cutting edge, the cutting edge comprising two leading portions and two trailing portions.

Preferably the cutting edge comprises two leading portions and two trailing portions, said cutting edge being formed by two laser cuts, each laser cut forming one notch in the tubular body at the cutting end.

Preferably the cutting edge is formed by a single arcuate laser cut, wherein the laser cutting beam is rotated about 180 degrees of the circumference of tubular punch body, the beam passing through a first adjacent wall portion of the tubular punch body and a second opposite wall portion, and wherein the laser cutting beam acts perpendicular or near perpendicular to the surface of the tubular punch body.

Preferably the profile of the cutting edge is of a saddle shape.

Preferably the forming of the cutting edge comprises cutting a saddle cut of material away from the tubular punch body at the cutting end.

Preferably the forming of the cutting edge comprises cutting a fishmouth cut of material away from the tubular punch body at the cutting end.

Preferably the tubular body is of a diameter under 10 mm.

Preferably the tubular body is of a diameter between 2 mm and 5 mm.

Preferably the tubular body is of a diameter between 2.5 mm and 3.5 mm.

Preferably the wall thickness of the tubular body is less than 15% of the diameter of the tubular body.

Preferably the wall thickness of the tubular body is less than 10% of the diameter of the tubular body.

Preferably the wall thickness of the tubular body is between 5% and 10% of the diameter of the tubular body.

Preferably the wall thickness of the tubular body is between 0.1 mm and 1 mm.

The term “axis” as used in this specification means the axis of revolution about which a line or a plane may be revolved to form a symmetrical shape. For example, a line revolved around and axis of revolution will form a surface, while a plane revolved around and axis of revolution will form a solid.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

Preferred embodiments of the invention will be described by way of example only and with reference to the drawings, in which:

FIGS. 1A and 1B show views of a punch of the present invention as part of a punch assembly.

FIG. 2A shows a side view of the punch and punch assembly of FIGS. 1A and 1B.

FIG. 2B shows a section view of the punch and punch assembly of FIG. 2A.

FIGS. 3A and 3B show the punch and punch assembly of FIGS. 1-2 being driven into an element of tissue.

FIGS. 4A and 4B show a punch according to one embodiment.

FIGS. 5A and 5B show a punch according to another embodiment.

FIGS. 6A and 6B show a punch according to another embodiment.

FIGS. 7A and 7B show a punch having a single leading portion according to another embodiment.

FIGS. 8A and 8B show a punch according to another embodiment.

FIGS. 9A and 9B show two punches configured to have cutting edge of differing depths and profiles.

FIG. 10 shows a punch having a cutting edge according to another embodiment wherein there are a plurality of leading and trailing portions.

FIGS. 11A and 11B show a side view and cross section view respectively of a punch according to one embodiment.

FIG. 12 shows a punch having a cutting edge comprising a plurality of serrations or burrs.

FIG. 13 shows a punch having a cutting edge comprising a plurality of serrations or burrs.

FIG. 14 shows a punch having a cutting edge wherein only the boundary with the side walls of the tubular body comprise burrs or serrations.

### DETAILED DESCRIPTION OF PREFERRED EMBODIMENTS

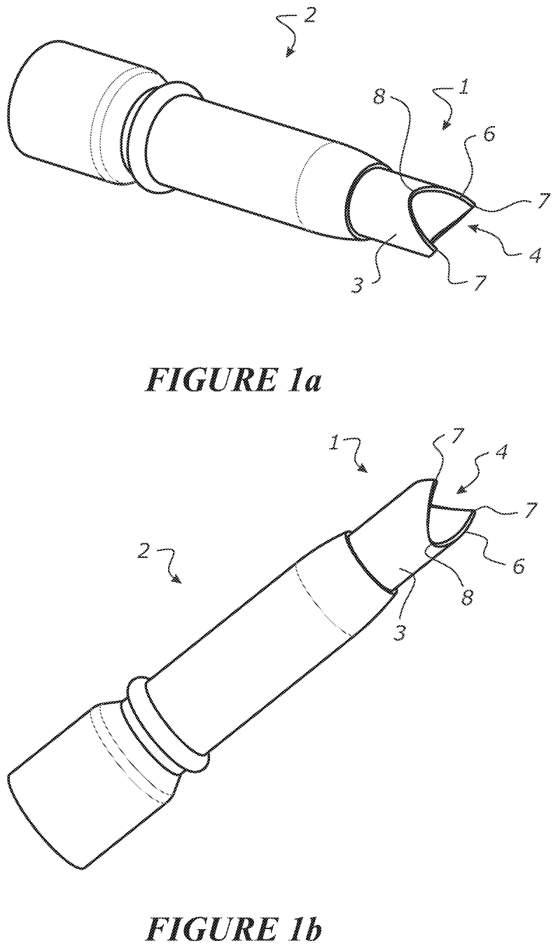

With reference to FIGS. 1A and 1B, two views of a punch 1 are shown as part of a punch assembly 2. The punch 1 may be provided as part of the punch assembly 2 in certain embodiments to enable or assist the punching of a sample by the punch 1. For example, the punch assembly 2 may couple with a driving element (not shown) at the end distal of the punch assembly 2, in order to drive the punch 1 into and/or through an element to be sampled.

In a preferred embodiment the punch 1 is a punch for taking samples of animal tissue, where tissue means any part of a living thing, particularly any part made up of similar cells, or any part or parts that perform a similar function. The punch 1 may be used to cut, tear or otherwise remove a sample of such tissue from an animal by the action of the punch against the tissue to be sampled. The cut tissue sample is preferably then retained within the punch for storage, testing, analysis, or other purposes.

As seen in FIGS. 1A and 1B, the punch 1 comprises a tubular punch body 3 having a cutting end 4 and a free end 5. The cutting end 4 further comprises a cutting edge 6. The cutting edge 6 is formed from the tubular punch body at the cutting end 4 so as to have a perimeter greater than the circumference of the tubular punch body. Accordingly, the cutting edge 6 comprises at least one leading portion 7 and at least one trailing portion 8.

In a preferred form, shown in FIGS. 1A and 1B, the cutting edge of the punch comprises two leading portions 7 and two trailing portions 8. In the embodiment of FIGS. 1A and 1B, the leading portions and trailing portions and the cutting edge between each portion is preferably configured such that the profile of the cutting edge 6 is in the form of a saddle cut or fish-mouth cut.

During the cutting of a tissue sample, the cutting edge 6 is pressed or driven into or preferably through the tissue to be sampled. The cutting edge 6 separates the sample from the animal and causes the cut sample to be passed within the bore of the tubular punch body.

During cutting of a tissue sample by a punch, it is possible that the tissue may become lodged or stuck against the cutting end, or caught by it, and remain at least partly covering or outside of the cutting edge 6, rather than passing into the bore of the tubular punch body 3. This may commonly occur because the cutting edge is unable to cut the tissue proximate to it. If a sample gets stuck on the cutting end of the punch, it may clog the punch so as to prevent the sample from being stored, or may jam other external components of a sampling device with which the punch may be used during sampling.

In order to prevent clogging of the punch in such a manner, prior art punches have commonly involved the sharpening of the cutting edge 6 in order to present a sharpened surface to the tissue, which may then more easily cut or shear through the tissue to prevent clogging. However, sharpening of the cutting edge, such as by grinding of the tube surface, may be costly and time consuming, and may still result in a surface having a cutting edge prone to clogging of tissue samples.

It is contemplated that the punch 1 of the present invention may at least partially address the issues associated with clogging of punches and improper cutting of tissue to be sampled, without requiring any post-formation sharpening of the cutting edge.

With reference to FIGS. 1A and 1B further details of the punch 1 of the present invention and its cutting edge 6 will now be described. The at least one leading portion 7 of the cutting edge 6 preferably comprises a peak or point, configured to puncture or pierce the surface of the tissue upon operation of the punch. The profile of the cutting edge 6 between the at least one leading portion and at least trailing portion then allows progressive cutting of the tissue sample about the circumference of the tubular punch body as the punch is operated into the tissue. The profile of the cutting edge, comprising at least one leading portion 7 and at least one trailing portion 8, operates to progressively cut the tissue by creating zones of increased pressure at the leading portion, then gradually introducing the remainder of the cutting edge 6 as the punch travels through the tissue.

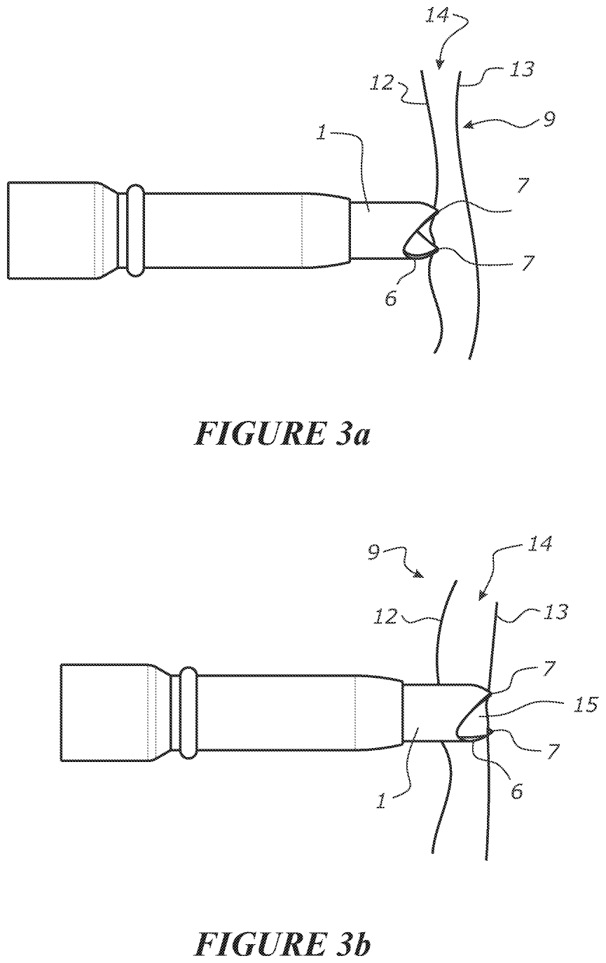

As seen with reference to FIGS. 3A and 3B, the cutting edge 6 is presented oblique to the surface of tissue to be sampled 9 for at least a majority of the portion of the cutting edge between a leading portion 7 and a trailing portion 8. The oblique angle of this portion of the cutting edge may preferably act to both progressively engage the cutting edge 6 with the tissue to be sampled 9 and to increase the shearing effect of the cutting edge 6 against the tissue 9.

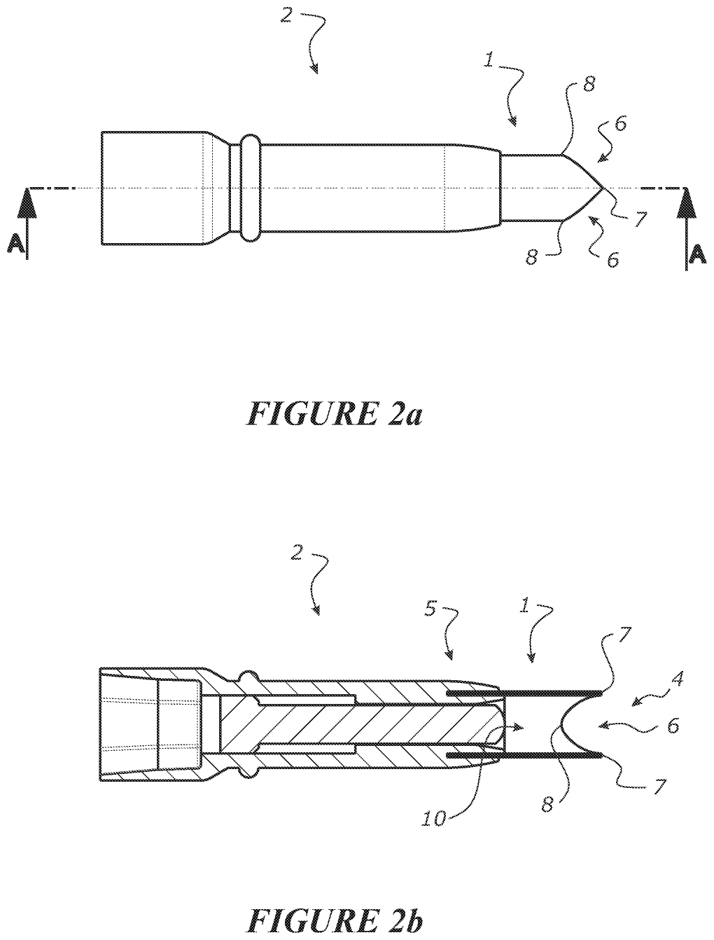

FIGS. 2A and 2B show a side view and section view respectively of the punch and punch assembly of FIGS. 1 and 2, thus showing two perpendicular views of the punch 1 and its cutting end 4. In the embodiment of FIGS. 2A and 2B, the cutting edge 6 comprises two leading portions 7 and two trailing portions 8. As seen in the view of FIG. 2A, a single leading portion 7 is visible and two trailing portions 8 are visible. In the respectively perpendicular section view of FIG. 2B, taken along the line A-A of FIG. 2A, both leading portions 7 are visible along with a single trailing portion 8.

Although the cutting edge 6 between the leading portion 7 and trailing portions 8 seen in FIG. 2A appear to be substantially straight in their profile, due to the tubular nature of the punch body 3 a curved cutting edge profile is provided between the leading portions 7 and trailing portions 8, as seen in FIG. 2B.

Even where the cutting edges shown in FIG. 2A have a profile of arcuate shape cutting edge profile from the perspective of FIG. 2B will be curved.

Also shown in FIG. 2B is the bore of the tubular punch body and sample holding cavity 10.

The cutting edge 6 is formed from the cutting end 4 of the tubular punch body 3. This forming may be by way of laser cutting, waterjet cutting, plasma cutting, mechanical cutting, or other commonly used forms of cutting. In the preferred embodiment the forming is by laser cutting.

The formation of the cutting edge 6 is preferably by at least one cut using one of the aforementioned cutting methods to remove material from the tubular punch body at the cutting end. It is contemplated that the type of cutting method and direction of such cuts may directly influence the nature of the cutting edge, and consequently the performance of the punch during cutting of a tissue sample.

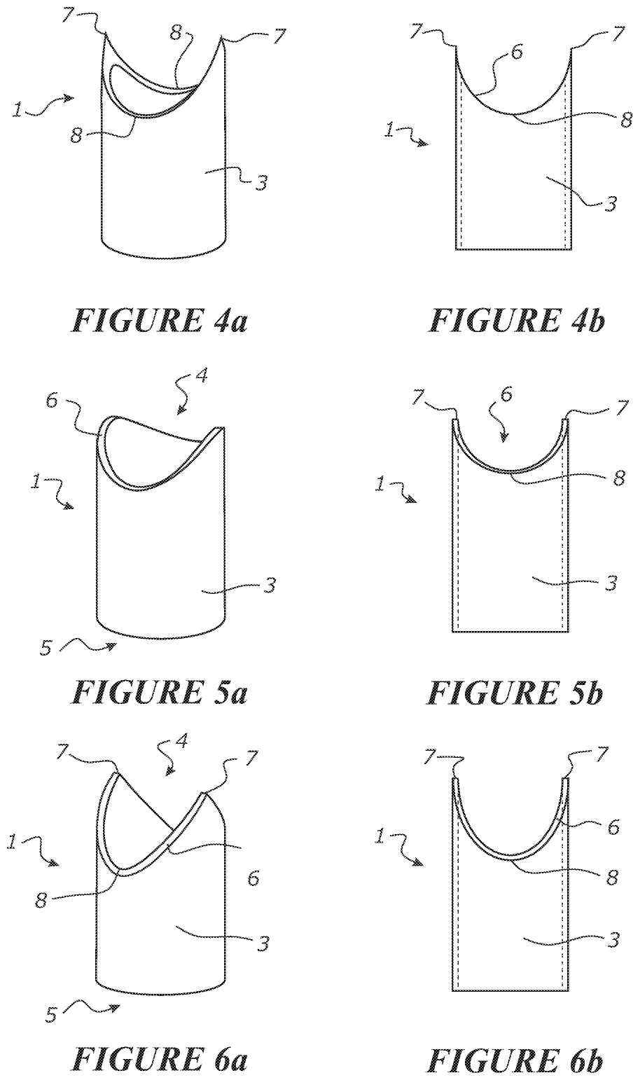

FIGS. 4A and 4B show a punch 1 and its cutting edge 6 under one cut formation configuration. A cut or series of cuts have been directed in a linear manner relative to the tubular body 3 of the punch, in order to cut away a U shape of material from the tubular punch body. The removal of this U shape of material from the tubular body results in a saddle-shape profile of the cutting end 4, with two leading portions 7 and two trailing portions 8. Each of the trailing portions 8 and leading portions 7 respectively are opposed from each other about the circumference of the tubular body.

Due to the linear direction of the cut relative to the tubular body and the fact the cut is made across the entire width of the tubular body, the wall thickness of the tubular punch body decreases towards the leading portions 7. This may cause an increased sharpness or pointedness in the profile of the leading portion 7, and may consequently enable the punch to more easily puncture the surface of tissue to be sampled. However, the thinning of the wall of the tubular body from the trailing portions 8 towards leading portions 7 may come at a cost of decreased strength of the cutting edge towards the leading portions, and the thinned regions of the wall of the punch body may be susceptible to bending or breaking off.

The cutting edge 6 of the punch 1 shown in FIGS. 4A and 4B may be formed by a plurality of cuts, or preferably, particularly where the formation of the cutting edge is by laser cutting, by a single cut passing through the entirety of the tubular punch body. For example, a cut may be directed in a U shape perpendicular to the tubular punch body, as is shown in FIG. 4B, such that the cut passes through both the immediate and opposite wall portions of the tubular punch body.

In some embodiments it may be desirable to provide a cutting edge having constant wall thickness of the tubular punch body about its perimeter. An example of a punch and cutting edge 6 formed in such a way is shown in FIGS. 5A and 5B. The cutting edge shown in FIGS. 5A and 5B may be formed by a cut directed rotationally around the tubular body, and at a perpendicular angle to the surface of the tubular body.

Such a cutting edge may be formed by the rotation of the cutting element about the tubular body, or the rotation of the tubular body relative to the cutting element. It may further be formed by a series of discrete cuts, or one continuous cut about the circumference of the tubular body.

It is specifically contemplated that the cutting edge 6 shown in FIGS. 5A and 5B may be formed by a single cut, such as by a laser cut, waterjet cut, or plasma cut, which passes through both the proximate and adjacent side wall portions of the punch body in a single cut. Provided the cut passes through both sides of the tubular punch body, the cutting edge 6 may be formed by a single rotation of the cutting element relative to the tubular body about the tubular body of 180 degrees.

In a preferred embodiment the formation of the cutting edge is by the rotation of a laser cutter element about the circumference of the tubular body, where the laser cutter only passes through the proximate side wall portion not both the proximate and opposite side wall portions, such that the forming involves a rotation about the entire circumference of the tubular body.

Where the forming is by cutting through both the local and adjacent portions of the tubular punch body's wall in a single cut, and passing through a rotation of only 180 degrees, it is possible that the cutting edge 6 of a punch 1 may be formed more efficiently than were a cut directed through single side wall portion about 360 degrees of the circumference of the tubular body.

The constant side wall thickness about the cutting edge 6 shown in FIGS. 5A and 5B may be desired to provide a cutting edge having leading portions 7 for piercing or puncturing the tissue to be sampled, and a continuously varying cutting edge from the leading portions to the trailing portions 8, yet providing constant strength of the cutting edge about its perimeter due to the constant side wall thickness.

A further manner of cutting edge 6 of a punch 1 is shown in FIGS. 6A and 6B. The cutting edge 6 similarly to the cutting edge shown in FIGS. 4A, 4B, 5A and 5B comprises a saddle-like profile, having two leading portions 7 and two trailing portions 8. Rather than cutting a U-shape of material from the cutting end of the tubular body, or rotating a cut about the circumference of the tubular body, the cutting edge of FIGS. 6A and 6B may be formed by cutting a V-shape of material away from the cutting end of the tubular body 3.

While the cutting of the tubular body may not be directed at all points perpendicular to the surface of the tubular body, causing the cutting edge to have some sharpness in the longitudinal direction of the tubular punch body, in the preferred form the cutting edge is substantially blunt in the longitudinal direction of the tubular body.

However, regardless of the forming method used in the forming of the cutting edge of the punch, whether it be laser cutting, waterjet cutting, plasma cutting, mechanical cutting, or any other commonly used cutting method, the formed cutting edge preferably comprises a non-smooth surface finish. This surface finish preferably comprises one or more burrs or serrations, the burrs or serrations being formed incidental to the forming process.

For example, where the forming of the cutting edge is by laser cutting, the pulsing of the laser cutter as it passes through the material of the tubular body preferably results in the uneven surface finish. While the pulsing of a laser cutter may in some instances be at regular intervals, the burrs that are provided on the cutting edge may not be regularly spaced or sized, but rather may be somewhat random in location and dimensions.

During forming of the cutting edge, such as with a laser cutter, the cutting element may most readily be directed from the outside of the tubular punch body towards its interior. For example, the laser cutter may be directed onto the external face of the punch body, and cut through it to the internal face. The action of cutting from the outside face to the inside face preferably causes burrs or serrations due to forming to be disposed on or towards the part of the cutting edge adjacent the inside of the tubular punch body. In this configuration, the burrs or serrations may be particularly useful in cutting a sample of tissue such that it may be held within the bore of the tubular punch body.

A view of the punch 1 of the punch and punch assembly of FIG. 2A is shown in FIG. 11A. Shown on the cutting edge 6 are a plurality of burrs or serrations 11.

Similarly, FIG. 11B shows a view of the punch of the same configuration as shown in FIG. 2B, where the cutting edge 6 is shown to have the plurality of burrs or serrations 11.

The burrs or serrations 11 of the cutting edge 6 may act to cut or tear the tissue being sampled, in a similar fashion as a serrated knife cuts through a material.

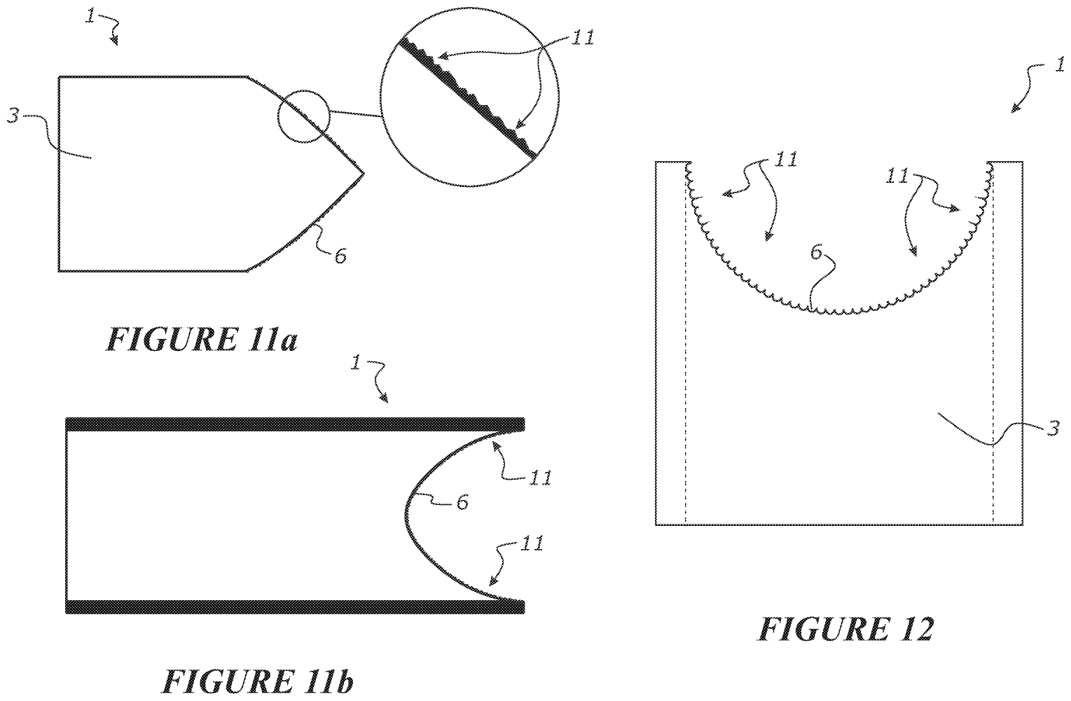

Shown in FIG. 12 is a side on view of a punch 11, showing the side wall thickness of the tubular body 3, wherein the cutting edge 6 comprises a plurality of burrs or serrations. In the particular configuration shown in FIG. 12, the burrs or serrations may be those formed by the pulsing of a cutter such as a laser cutter.

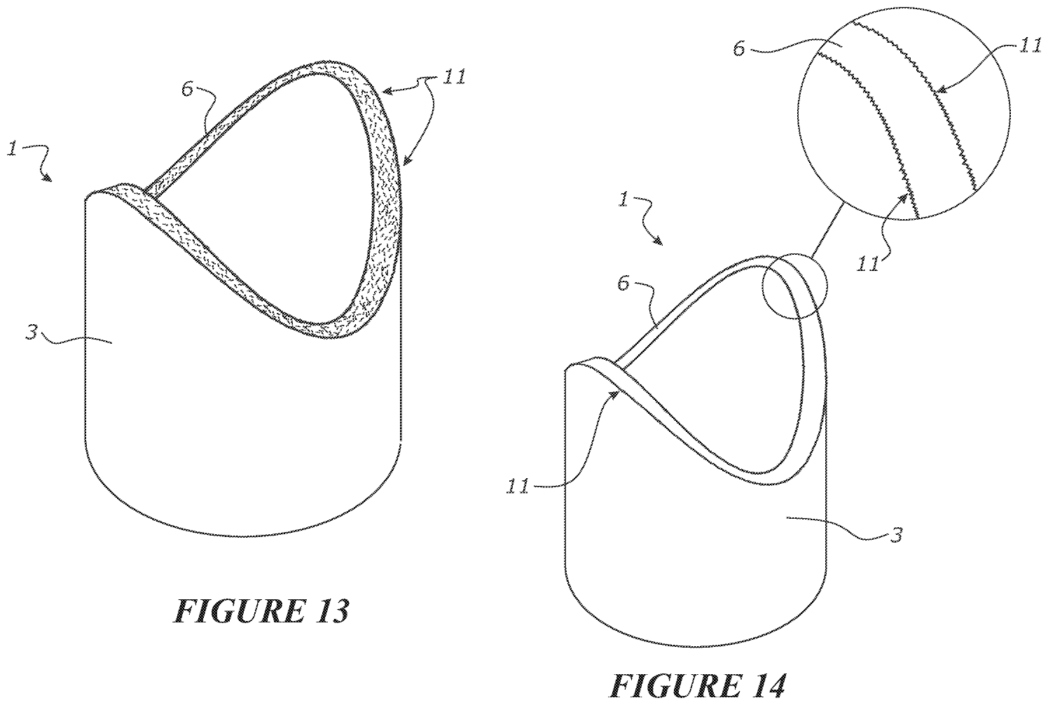

With reference to FIG. 13 a view of the punch of FIG. 5A is shown wherein the cutting edge 6 comprises a plurality of burrs or serrations 11. In this configuration, the burrs or serrations 11 are present both on the edge surfaces of the cutting edge 6, and on the main portion of the cutting surface, which lies between the two walls of the tubular body 3.

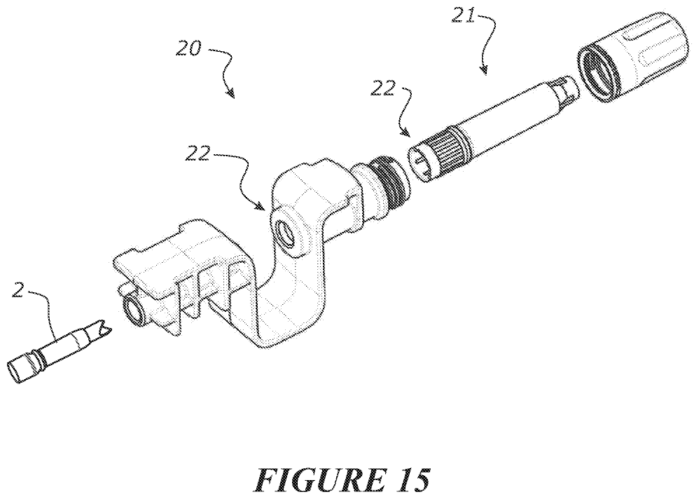

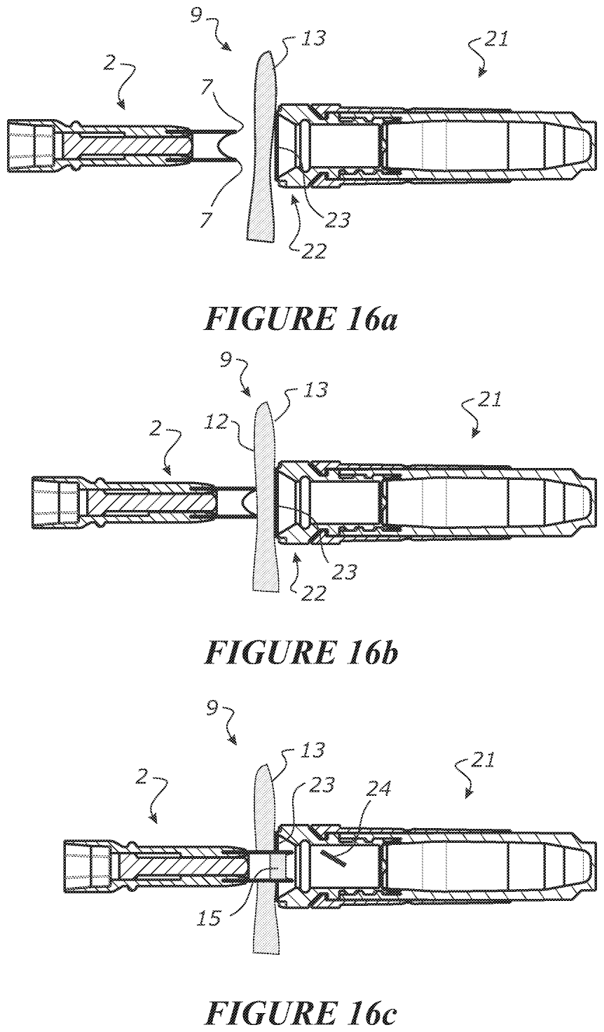

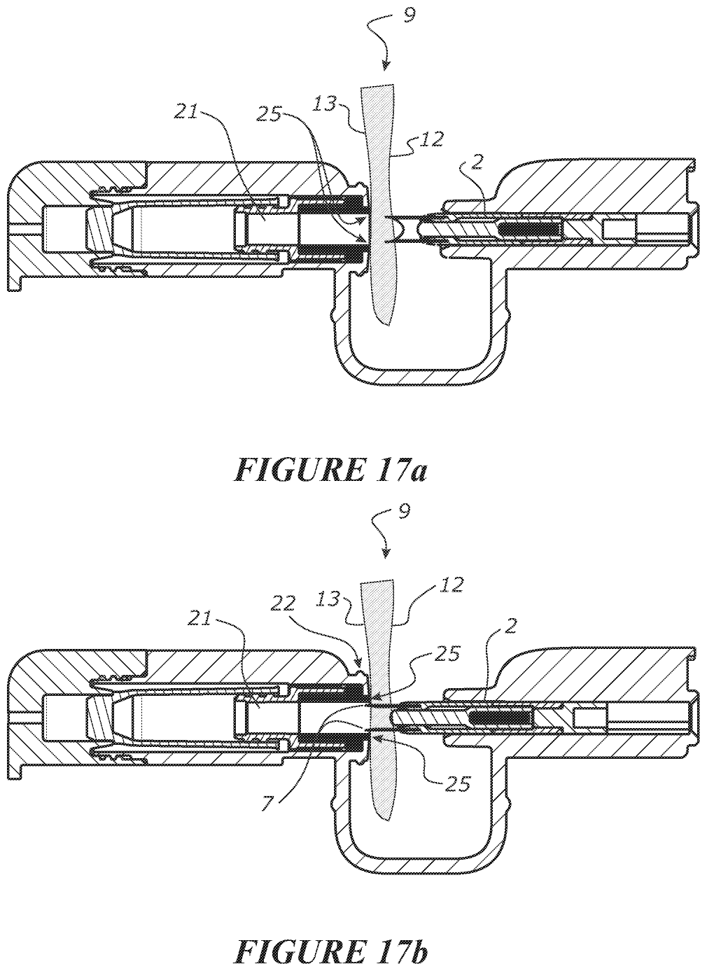

FIG. 14 shows further variation wherein the cutting edge 6 comprises burrs or serrations positioned only on the external portions of the cutting edge towards the side walls of the tubular body 3. For example, a V or near V shaped cut of material may be made from the tubular punch body, forming it to the shape shown in FIG. 2A. Each side of the V shaped profile may be formed by a plurality of cuts, either through only one or both side walls portions of the tubular punch body. Preferably however the cutting edge 6 along each side of the V shown in FIG. 2A, or the portion which is also the portion shown between the two leading portions 7 in FIG. 6A, is formed by a single cut directed into the page as shown in FIG. 2A.

The resulting cutting edge 6 may have some variance in thickness of the side wall of the tubular punch body about its perimeter, yet it retains the full wall thickness at the leading portions 7 as seen in FIG. 6B. While the leading portion 7 retain the full wall thickness of the tubular punch body, the V shaped cut may provide sharp peaks in the cutting edge at each leading portion. These peaks may assist in the initial puncturing or cutting of the tissue to be sampled.

As previously described, the cutting edge 6 of a punch according to the present invention are preferably formed by cutting of the material of the tubular body, and do not involve any post-formation sharpening. Accordingly, while the cutting edges may comprise one or more burrs or serrations, as have been previously described, they are largely unsharpened and blunt in the longitudinal direction of the tubular punch body.

This nature of the cutting edge 6 may render it naturally indisposed to punching by both a lateral movement of the punch into the tissue and a rotation of the punch body about its longitudinal axis. This is because the un-sharpened cutting edge surface, while it may present some burrs or serrations capable of tearing or cutting the tissue surface, under rotation will act largely directly against the surface of the tissue to be sampled. This manner of engagement does not enable smooth cutting of the tissue, but at best forced tearing or rupturing of the tissue across the width of the cutting edge.

Due to the presentation of the one or more leading portions 7 at the cutting end 4 of the punch, the punch is preferably configured for cutting under operation in a purely linear action, in the direction of the longitudinal axis of the punch body.

Under a linear operation, as shown in FIG. 3A, the leading portion or portions 7 first contact the tissue to be sampled 9. Due to their peaked nature, the leading portions are adapted to puncture or rupture any skin surface of the tissue, to allow the blade to come into contact with the bulk of the tissue. Under further operation of the punch into the tissue 9 the subsequent portions of the cutting edge 6 between the leading portion and trailing portion 8 progressively shear through the tissue, the width of the cut increasing as the punch travels into the tissue.

In some embodiments, as previously described, the cutting edge may have a parabolic or otherwise arc-shaped profile. Where such a profile is provided, the angle of the cutting edge to the tissue starts out most steeply angled at the leading portion or portions 7, and decreases towards the trailing portion or portions 8. Under the cutting of such a cutting edge 6 against an element of tissue to be sampled, the leading portion or portions 7 first pierce the tissue, then the following cutting edge cuts or shears the tissue at a steep incident angle. Subsequent portions of the cutting edge are then presented at progressively shallower incident angles, until the trailing portion or portions 8 are reached which are have a profile substantially parallel to the tissue being sampled.

Because the punch of the present invention has at least one leading portion and at least one following portion of its cutting edge, the cutting edge comprises a perimeter of greater length than the circumference of the tubular punch body. In preferred forms the configuration of the cutting edge and at least one leading and trailing portions is such that the cutting edge perimeter is between 20% and 35% longer than the circumference of the tubular punch body.

This increased cutting surface length, and the progressive nature of the entry of the punch into the tissue to be sampled, allow an increase in the cutting pressure exerted on the tissue being cut at any instant, for the same driving force on the punch. This increased pressure may translate to increased shear forces and increased cutting of the tissue.

Similarly, the increased cutting surface length and progressive nature of the entry of the punch may allow the same cutting or shearing pressures to be achieved at lower driving forces.

The effect of magnifying the cutting or shearing pressures comes at the cost of requiring a longer stroke of the punch in order to pass through the tissue material. The increase in the required stroke will be equal to the distance in the longitudinal direction of the punch between a leading portion and a trailing portion.

In preferred forms, the distance between the leading and trailing portions of the punch along the length of the longitudinal punch body will be equal to or less than the diameter of the punch body. More particularly, the distance between the leading portions and the trailing portions may be approximately equal to the radius of the punch body.

As previously described, the leading portion or portions of the punch preferably act to increase the pressure exerted on the tissue to be cut, for a given driving force of the punch, as the punch is first put into contact with the tissue to be sampled. This increase in pressure upon initial contact of the punch with the tissue preferably results in increased ability for the punch to puncture or cut into the tissue.

Commonly, tissue to be sampled, such as the ear of an animal, will comprise a layer of flesh covered on both sides by a skin layer, and the skin layers may commonly be covered with hair. In order for the punch to pass through the tissue to be sampled, for example an ear, the cutting edge of the punch must puncture the first skin surface, preferably cutting any proximate hairs, proceed through the central flesh and then puncture from the back-side the second skin surface on the opposite side of the ear.

During use of the punch the one or more leading portions of the cutting edge preferably act to first puncture or cut the first skin surface. As the punch is driven further, the leading portion continues into the flesh between the skin surfaces, and the following portions of the cutting edge progressively widen the initial puncture or punctures made by the leading portion or portions respectively. Once the punch driven a sufficient distance, the trailing portion(s) of the cutting edge will be engaged with the first skin surface to cut it, and a cut will have been effected about the entire circumference of the tubular punch body at the first skin surface.

The progression of the punch through the tissue will cause the leading portion(s) to reach the second skin surface on the far side of the ear. Further driving of the punch preferably causes the leading portions to break through the second skin surface in reverse, from the inside out. Due to the concentration of pressure at the leading portion(s), the punch is thought to able to puncture the skin surface at a lower driving force than would be possible with an identical cutting edge which presents the entire cutting edge to the skin surface simultaneously.

Further driving of the punch results in a similar cutting action of the second skin surface as already described in relation to the first skin surface, such that the second skin surface is also cut about preferably the entire circumference of the tubular punch body.

The punching operation of the leading portions of one embodiment of the punch of the present invention is shown in FIGS. 3A and 3B. In FIG. 3A the leading portions 7 of the cutting edge 6 of the punch 1 are shown to be puncturing the first skin surface 12 of the tissue to be sampled 9. In FIG. 3B the punch has been driven further into the tissue to be sampled 9, so that the entire cutting edge 6 has cut the first skin surface 12 and has progressed into the intermediate tissue 14. As seen in FIG. 3B, the leading portions 7 of the punch are being engaged with and puncturing the second tissue surface 13. Upon further driving the cutting edge of the punch may be passed fully through the second skin surface 13, so that an element of tissue is cut away from the tissue to be sampled 9. The element of tissue, being the tissue sample, is preferably retained within the sample holding cavity 10 in the bore of the tubular punch body.

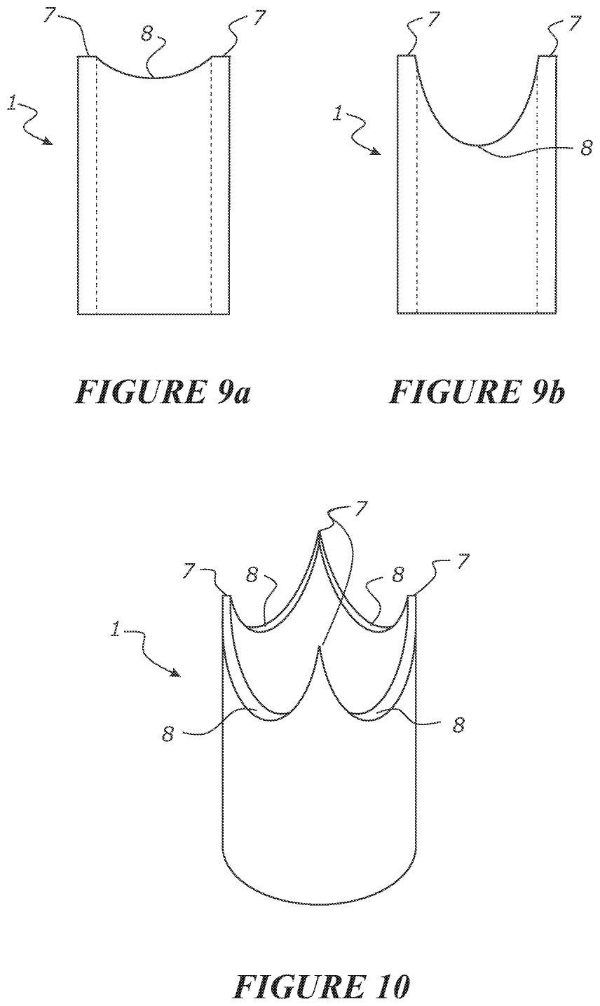

In order to suit different cutting characteristics or cutting requirements, such as for different types of tissue, the cutting edge 6 of a punch 1 may be formed having a range of different possible depths of cut between their leading portions 7 and trailing portions 8. A deeper cutting edge profile, as shown in FIG. 9B, may concentrate the pressures applied to the tissue in order to cut it, and provide relatively slower progressive engagement of the cutting edge with the tissue to be cut, but will require a longer travel of the punch to pass through the same depth of tissue as would a punch of comparatively shallower cutting edge depth, for example as shown in FIG. 9A.

In a preferred form, the depth of a cut between a leading portion and trailing portion along the length of the tubular punch body may preferably be approximately equal to the diameter of the tubular punch body. Alternatively, the depth of the cut may be proximately equal to the radius of the tubular punch body. More particularly, the angle between two cutting edge portions between a leading portion and the adjacent to trailing portions 8, for example as shown in FIG. 2A, may preferably be proximately equal to 90 degrees.

The punch of the present invention may particularly be suited for use with thin sections of tissue, for example between 3 mm and 5 mm in thickness, where the punch is passed through the entire thickness of the tissue.

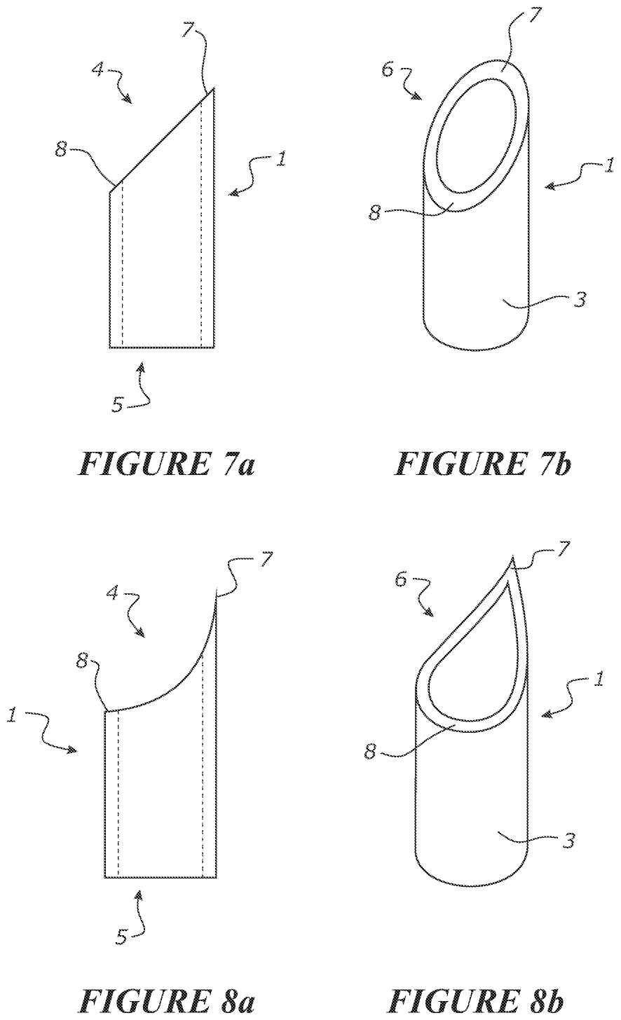

While in the preferred form the punch 1 comprises a cutting edge having two leading portions into trailing portions, other configurations of the punch are possible within the scope of the invention. For example, the punch 1 may have a cutting edge 6 having a single leading portion 7 and a single trailing portion 8. Two examples of such a configuration are shown in FIGS. 7A, 7B, 8A and 8B.

The cutting edge 6 of the punch 1 of FIGS. 7A and 7B are formed by a cut of the tubular punch body made at an angle non-perpendicular to the longitudinal direction of the tubular punch body. Such a cutting edge 6 may have a tube wall thickness that varies about its perimeter.

In the second configuration having a single leading portion 7 and single trailing portion 8, shown in FIGS. 8A and 8B, an arcuate cut is made across the tubular punch body 3. The resulting cutting edge 6 may have an increased narrowing of the wall thickness of its cutting edge at either or both of the leading portion 7 and trailing portion 8. As seen in FIGS. 8A and 8B, the arcuate cut forms a leading portion 7 having a significantly narrowing side wall thickness, resulting in a sharply peaked or pointed leading portion.

The punch 1 according to the present invention may comprise a plurality of leading portions 7 and trailing portions 8, as shown in FIG. 10. In the configuration of FIG. 10, the cutting edge 6 of the punch 1 comprises four leading portions 7 and four interspaced trailing portions 8. In other embodiments the punch may comprise an odd number of leading and trailing portions, such as three.

The cutting edge 6 according to the present invention has been described largely as having an arcuate profile between any two leading portions, or either side of a single leading portion. More particularly it has been described as a parabolic or continuously curved arcuate profile in relation to particular embodiments. It is contemplated that such profiles may have advantages over other non-continuously curved profiles or non-arcuate profiles, as a continuously varying surface profile may result in decreased likelihood of tissue to catch or snag on the cutting surface as it passes by. Particularly, cutting edge profiles having trailing portion(s) that are less deeply recessed or less sharply curved may present these advantages.

However other forms of the cutting edge profile may be provided within the scope of the invention, such as cutting edges of substantially discontinuously curved profiles.

The tubular punch body 1 may commonly be formed from a metal, such as a steel or a stainless steel. It may alternatively also be formed from other materials, such as plastics of suitable hardness or ceramics. It is preferred that the material used for the punch body, in combination with the forming method selected for forming the cutting edge, is such that the forming of the cutting edge results in a non-smooth surface finish. Preferably the non-smooth surface finish is a surface finish which is rougher than that of the external or internal surfaces of the tubular punch body, and preferably which comprises burring or serration.

In use in taking a tissue sample, the punch may commonly be used in combination with a die. This die may be a stand-alone element, or may be part of a storage container for the cut tissue sample and/or punch, such as a sample tube. In such a configuration the die may comprise the opening of the storage container or sample tube.

In one possible configuration, the punch assembly 2 may be provided for use along with a sample tube 21 as part of a cartridge 20. As shown in the exploded view of FIG. 15, the punch assembly 2 and sample tube 21 may be housed within such a cartridge 20, and axially aligned with each other in the cartridge for use. As shown in FIG. 15, either the opening of the cartridge 20 at the side of the sample tube 21 or the opening of the sample tube 21 itself may provide a die 22 for use with the punch assembly in cutting a tissue sample.

With reference to FIGS. 16a-c a first configuration of the interaction of a punch assembly 2 and die 22 in cutting a tissue sample 15 is shown. Seen in FIG. 16a is a punch assembly 2 aligned with a die 22, which happens to be provided as part of a sample tube 21. The die 22 in this configuration comprises an opening for receiving at least the punch body 3 of the punch assembly 2. The die 22 further comprises a perforable support element 23. The perforable support substantially covers the opening of the die 22, such that it may provide back-support to the second tissue surface 13 of the tissue 9 in cutting of the tissue sample 15.

The perforable support element 23 may preferably be in the form of a cap or seal extending across the opening of the die 22. The perforable support 23 may be of an either substantially stiff or rigid material, or may comprise a flexible material. For example, it may be formed by a hard plastic, or a soft plastic such as thermoplastic polyurethane (TPU). In the latter configuration, the perforable support may have elastic characteristics, such that it may provide back-support to the second tissue surface 13, yet flex under the driving force of the punch assembly 2 towards the die 22 during sampling.

Regardless of the material configuration and either substantially rigid or elastic nature of the back-support provided by the perforable support 23, the perforable support must be able to be perforated by the punch during sampling. Furthermore, while in this embodiment it is desirable to provide back-support to the tissue being sampled, it is simultaneously desirable that the perforable support 23 does not significantly inhibit the driving of the punch assembly 2 to complete the cutting of a tissue sample 15, and in the configuration of FIG. 16a-c, lodge the punch assembly within the sample tube 21. This means that the perforable support 23 should also be readily perforable, in order that it does not significantly inhibit the forward progress of the punch assembly 2.

It is desirable that the configuration where the die 22 comprises a perforable support 23 the cutting of a tissue sample 15 and perforation of the perforable support 23 may be provided without requiring any significant increase in the driving force of the punch assembly 2. Thus in at least some embodiments, the configuration of the perforable support 23 will be such that it may provide at least some back-support to the second tissue surface 13, yet will readily be perforated by the punch.

To this end, the punch of the present invention with its at least one leading portion 7 may provide advantages over cutting of such a perforable support 23 with a punch comprising only a single continuous edge with no leading portion. Similarly to as has been described previously in relation to the operation of the punch of the present invention in cutting a tissue sample, the at least one leading edge 7 may serve to initially pierce the perforable support 23, then progressively cut the perforable support 23 about the remainder of the circumference of the punch body 3 as the punch moves past the perforable support.

The various leading portion and cutting edge configurations as previously described, including those resulting from the proposed methods of manufacture of the punch body, may similarly provide advantages in the cutting of a perforable support 23. These advantages may mean that a relatively thicker, tougher, or more rigid perforable support may be able to be used in combination with the punch of the present invention. This may provide for an increased degree of back-support of the tissue 9 during sampling, without a consequential increase in the forces required to perforate the perforable support 23.

Interposed between punch and sample tube of FIG. 16a is an element of tissue to be sampled 9. As seen in FIG. 16b, the punch assembly 2 has been driven towards the die 22 and the leading portions 7 of the punch have made contact with the tissue 9 and the first tissue surface 12. The tissue 9 has been forced against the perforable support 23, and the second tissue surface 13 is supported against the perforable support 23.

Under the back-support of the perforable support 23 the cutting of the tissue by the punch may be improved, allowing the punch to take a cleaner and more continuous sample. Without the back-support provide by the perforable support 23, it is possible for the un-cut tissue 9 to be driven at least partially into the opening of the die 22. When driven into the die, the portions of the un-cut tissue 9 lateral of the punch may be folded over about the leading edges of the punch. The effect of this non-back-supported cutting may be to cause the tissue 9 to jam in the die 22 or otherwise inhibit the forward progress of the punch through the die 22. Alternatively, it may not jam the punch but the dragging of the tissue into the die 22 may result in poor cutting performance, particularly of the second skin surface 13.

Tissue, such as from a not cleanly cut second skin surface 13, which is drug into the die may end up between the outside of the punch and the die. This may result in undesirable biological contamination emanating from the punch and die, or punch and sample tube, or may mean that a punch assembly cannot fully seal against the die or sample tube. This may allow external contaminants in which may foul the tissue sample.

In the configuration of FIGS. 16a-c, the tissue 9 remains supported by the perforable support 23 as the cutting edge of the punch assembly 2 cuts the tissue. Once the at least one leading portion 7 of the punch contacts the perforable support 23, it begins to perforate it.

A completed cutting of a tissue sample is shown in FIG. 16c. Here the punch assembly 2 has fully cut the tissue sample 15 from the tissue to be sampled 9, and has also fully perforated the perforable support 23 leaving a cut portion 24.

An alternative configuration of the use of the punch of the present invention with a die 22 is shown in FIGS. 17a-b. In this configuration, the die 22 does not comprise a perforable support 23 but rather comprises a substantially open die mouth. In some configurations a small seal may be used to seal the die mouth to prevent external contaminants entering through the die prior to sampling, but such a seal would not be such that it is able to provide any appreciable back-support to the tissue 9.

However, as previously described, it may still be desirable to provide for some degree of back-support of the tissue in order to at least in part ameliorate the identified disadvantages of dragging of tissue into the die, jamming, contamination, or inadequate sealing. In the configuration of FIGS. 17a-b, the back-support of the tissue, and particularly the second skin surface 13, are provided by the perimeter edges 25 of the die 22 itself.

Unlike in the configuration of FIGS. 16a-c, the die 22 of FIG. 17a-b presents an opening having a diameter closely corresponding to that of the punch 2. By this means, during cutting of a sample as seen in FIG. 17a, the tissue 9 and particularly second tissue surface 13 are supported adjacent the leading portions 7 by the perimeter edges 25. Such a configuration may at least in part ameliorate the above identified disadvantages of normal punch and die cutting, without requiring the presence of a perforable support 23 which must also be cut by the punch during sampling.

While there may be some bulging of the tissue at the center of the die opening into the die opening, because the second tissue surface 13 is supported directly adjacent the location of the cutting of the tissue by the punch, the cutting of the sample may be improved. Furthermore, the decreased gap between the outer perimeter of the punch 2 and the inside of the die 22 may also act to prevent tissue from being able to be dragged between them, and facilitate its being cut by the punch.

In addition to the back-support provided by the edges 25, the close proximity of the edges 25 to the cutting edge of the punch may furthermore provide a shear action between the two components. Such a shear action may also assist in the cutting of a tissue sample.

While described in configurations comprising either a perforable support 23 or the relative punch and die dimensions of FIGS. 17a-b, embodiments within the scope of the invention may comprise either of these configurations, both configurations in one, or other configurations of die for use with the tissue sampling punch.

While illustrated in FIGS. 15-17 in the configuration as part a sample tube 21 and/or cartridge 20, the punch of the present invention may be used with a die 22 where no such sample tube or cartridge are associated with it. While the foregoing provides description of a potential use of the punch of the present invention with a die 22, the punch may be used independent of any such die, such as by shooting the punch assembly through the tissue to be sampled, or by taking a punch-and-withdraw sample of an item of tissue where the punch does not pass entirely through the tissue.

1. A tissue sample punch to puncture into and through tissue, the punch comprising
a tubular body having a tissue puncturing end and a trailing end, the tissue puncturing end being formed to define saddle cut having at least two peaks that define the distal extent of the puncturing end.

2. A tissue sample punch comprising a tubular punch body having a cutting end and a free end, the cutting end comprising a cutting edge formed and having a perimeter greater than the circumference of the tubular punch body at the cutting end, the cutting edge presenting at least one leading portion and one trailing portion.

3. A tissue sample punch as claimed in claim 2, wherein the cutting edge at a leading portion comprises a peak in the profile of the cutting edge flanked by wake edges that are at an angle between 40 degrees and 120 degrees to each other.

4. A tissue sample punch as claimed in either of claim 2 or 3, wherein the bend of the cutting edge at the leading portion is a sharp bend rather than a curved bend.

5. A tissue sample punch as claimed in any one of claims 2 to 4, wherein the cutting edge comprises one leading portion, and the cutting edge varies continuously in an axial direction of the tubular punch body from one side of the leading portion to the trailing portion and back to the other side of the leading portion.

6. A tissue sample punch as claimed in any one of claims 2 to 4, wherein the cutting edge comprises multiple leading portions, and the profile of the cutting edge preferably varies continuously in an axial direction of the tubular body between any two adjacent leading portions.

7. A tissue sample punch as claimed in either of claim 5 or 6, wherein the continuous variation of the cutting edge acts to prevent the tissue being sampled from being caught or snagged on the cutting edge, particularly where the tissue comprises a skin membrane or surface hairs.

8. A tissue sample punch as claimed in any one of claims 2 to 7, wherein the forming of the cutting edge of the punch is by one of laser cutting, water jet cutting, plasma cutting and mechanical cutting.

9. A tissue sample punch as claimed in any one of claims 2 to 7, wherein the forming of the cutting edge of the punch is by laser cutting.

10. A tissue sample punch as claimed in claim 9, wherein the surface finish following the forming by laser cutting comprises a non-continuous surface profile.

11. A tissue sample punch as claimed in claim 9 or 10 wherein the pulsing of the laser during the laser cutting results in the formation of the non-continuous surface profile.

12. A tissue sample punch as claimed in any one of claims 7 to 11, wherein the forming of the cutting edge of the punch causes the cutting edge to comprise a plurality of burrs or serrations.

13. A tissue sample punch as claimed in claim 12, wherein the cutting edge is not sharpened or de-burred from its post-forming surface finish.

14. A tissue sample punch as claimed in any one of claims 2 to 13, wherein the tubular punch body at the cutting end comprises a metallic material which presents at the cutting edge, subsequent to its forming, a surface finish of greater roughness than the surface finish of the internal and external faces of the tubular punch body.

15. A tissue sample punch as claimed in claim 14, wherein the surface finish acts, during the cutting of a sample, to tear or rip adjacent portions of the tissue to be sampled.

16. A tissue sample punch as claimed in claim any one of claims 8 to 15, wherein the forming of the cutting edge comprises cutting at least one V or U shape of material away from the tubular punch body at the cutting end.

17. A tissue sample punch as claimed in any one of claims 8 to 15, wherein the forming of the cutting edge comprises cutting a saddle cut of material away from the tubular punch body at the cutting end.

18. A tissue sample punch as claimed in any one of claims 8 to 15, wherein the forming of the cutting edge comprises cutting a fishmouth cut of material away from the tubular punch body at the cutting end.

19. A tissue sample punch as claimed in any one of claims 2 to 18, wherein during the cutting of a tissue sample the cutting edge of the punch makes a first incision at the at least one leading portion from which the cutting edge is progressively engaged with the sample to be cut towards the at least one trailing portion.

20. A tissue sample punch as claimed in any one of claims 2 to 19, wherein between each at least one leading portion and at least one adjacent trailing portion the cutting edge that penetrates the tissue has a normal or normals that extends at a non-zero angle to the normal of the surface of the tissue it is cutting through.

21. A tissue sample punch as claimed in claim 20, wherein the cutting edge between each at least one leading portion and at least one adjacent trailing portion causes a concentration of shear forces on the tissue during the cutting of a tissue sample by the punch.

22. A tissue sample punch as claimed in claim 2, wherein the cutting edge is formed by at least one laser cut through at least one wall of the punch body made linearly relative to the tubular body, such that wall thickness of the tubular body varies about the cutting edge.

23. A tissue sample punch as claimed in claim 22, wherein the cutting edge comprises two leading portions the and the wall thickness of the tubular body at the cutting edge narrows towards each leading portion.

24. A tissue sample punch as claimed in claim 2, wherein the cutting edge is formed by at least one laser cut directed through a wall of the tubular body and perpendicular to the surface of the tubular body, such that tube wall thickness is continuous about cutting edge.

25. A tissue sample punch as claimed in claim 2, wherein the cutting edge is formed by at least one laser cut through a wall of the tubular body and directed at a constant angle to a central axis of the tubular body such that the formed cutting edge is at a constant angle relative to the wall of tubular body.

26. A tissue sample punch as claimed in claim 2, wherein the cutting edge is formed by a single arcuate laser cut, wherein the laser cutting beam is rotated about 180 degrees of the circumference of tubular punch body, the beam passing through a first adjacent wall portion of the tubular punch body and a second opposite wall portion, and wherein the laser cutting beam acts perpendicular or near perpendicular to the surface of the tubular punch body.

27. A tissue sample punch as claimed in any one of claims 2 to 26, wherein the tubular body is of a diameter under 10 mm.

28. A tissue sample punch as claimed in any one of claims 2 to 26, wherein the tubular body is of a diameter between 2 mm and 5 mm.

29. A tissue sample punch as claimed in any one of claims 2 to 26, the tubular body is of a diameter between 2.5 mm and 3.5 mm.

30. A tissue sample punch as claimed in any one of claims 2 to 29 wherein the wall thickness of the tubular body is less than 15% of the diameter of the tubular body.

31. A tissue sample punch as claimed in any one of claims 2 to 29, wherein the wall thickness of the tubular body is less than 10% of the diameter of the tubular body.

32. A tissue sample punch as claimed in any one of claims 2 to 29, wherein the wall thickness of the tubular body is between 0.1 mm and 1 mm.

33. A tissue sample punch as claimed in any one of claims 2 to 32, wherein the tissue to be sampled comprises the biological matter of one of an animal or a plant.

34. A method of manufacturing a tissue sample punch, the method comprising the steps of
providing a tubular body,
forming a cutting edge by making at least one cut through the wall of the tubular body,
wherein the forming is in a manner so as to cause burring or serration of the at least one cut and otherwise unsharpened cutting edge to remain intact.

35. A method of manufacturing a tissue sample punch as claimed in claim 34, wherein the forming of the cutting edge is by at least one cut of one of a laser cutter, water jet cutter, plasma cutter, or mechanical cutter.

36. A method of manufacturing a tissue sample punch as claimed in claim 35, wherein the forming of the cutting edge by at least one cut is such as to provide a cutting edge comprising a plurality of burrs or serrations.

37. A method of manufacturing a tissue sample punch as claimed in claim 36, wherein the burrs or serrations are of a size less than one tenth of the diameter of the tubular punch body.

38. A method of manufacturing a tissue sample punch as claimed in any one of claims 34 to 37, wherein the step of forming the cutting edge comprises no sharpening or de-burring of the cutting edge, other than any such sharpening or de-burring incidental to the at least one cut.

39. A method of manufacturing a tissue sample punch as claimed in any one of claims 34-38, wherein the step of forming the cutting edge comprises making at least one cut such that the wall thickness of the tubular body narrows towards each leading portion.

40. A method of manufacturing a tissue sample punch as claimed in any one of claims 34-38, wherein the step of forming the cutting edge comprises making at least one laser cut through at least one wall of the punch body, the cut laser being made linearly relative to the tubular body, such that wall thickness of the tubular body varies about the cutting edge.

41. A method of manufacturing a tissue sample punch as claimed in any one of claims 34-38, wherein the step of forming the cutting edge comprises making at least one laser cut directed through a wall of the tubular body and perpendicular to the surface of the tubular body, such that tube wall thickness is continuous about cutting edge.

42. A method of manufacturing a tissue sample punch as claimed in claim 41, wherein during the step of forming the cutting edge the laser cutting beam remains perpendicular or near perpendicular to the surface of the tubular body.

43. A method of manufacturing a tissue sample punch as claimed in any one of claims 34-42, wherein the step of forming the cutting edge comprises making at least one laser cut through a wall of the tubular body and such that the laser cutting beam is directed at a constant angle to a central axis of the tubular body, such that the formed cutting edge is at a constant angle relative to the wall of tubular body.

44. A method of manufacturing a tissue sample punch as claimed in any one of claims 34-43 wherein the step of forming of the cutting edge comprises cutting a fishmouth cut of material away from the tubular punch body at the cutting end.

45. A method of manufacturing a tissue sample punch as claimed in any one of claims 34-43 wherein the step of forming the cutting edge comprises making a single arcuate laser cut, wherein the laser cutting beam is rotated about 180 degrees of the circumference of tubular punch body, the laser cutting beam passing through a first adjacent wall portion of the tubular punch body and a second opposite wall portion, and wherein the laser cutting beam acts perpendicular or near perpendicular to the surface of the tubular punch body.

46. A kit of parts for use in taking a sample of biological matter comprising the tissue sampling punch of any one of claims 1 to 33 and a die, the die comprising an opening for receiving at least part of the tissue sampling punch.

47. The kit of parts of claim 46, wherein the die comprises a perforable support provided across the opening of the die, the perforable support able to provide support to the biological matter during cutting by the tissue sampling punch yet able to be perforated by the cutting edge of the punch to allow the receipt of at least part of the tissue sampling punch by the die.

48. The kit of parts of one of claim 46 or 47, wherein the die comprises an opening having an internal diameter closely corresponding to the outer diameter of either or both of the punch assembly or the punch body.

49. The kit of parts of claim 48, wherein the perimeter of the opening of the die acts in shear with the cutting edge of the punch body to cut a tissue sample.

50. The kit of parts one of claim 48 or 49, wherein the difference between the internal diameter of the opening of the die and the external diameter of either or both of the punch assembly or the punch body is less than 1 mm.

51. The kit of parts one of claim 48 or 49, wherein the difference between the internal diameter of the opening of the die and the external diameter of either or both of the punch assembly or the punch body is less than 0.5 mm.
